(19) United States
(12) Patent Application Publication
(10) Pub. No.: US 2021/0154230 A1
(43) Pub. Date: May 27, 2021

(54) INTRADISCAL T-REGULATORY CELL ADMINISTRATION FOR TREATMENT OF DISC DEGENERATIVE DISEASE

(71) Applicant: SpinalCyte, LLC, Houston, TX (US)

(72) Inventors: Pete O'Heeron, Houston, TX (US); Thomas Ichim, San Diego, CA (US)

(21) Appl. No.: 17/052,845

(22) PCT Filed: May 3, 2019
(86) PCT No.: PCT/US19/30564

§ 371 Date: Nov. 4, 2020

Related U.S. Application Data
(60) Provisional application No. 62/666,807, filed on May 4, 2018.

(51) Int. Cl. A61K 35/17; A61K 35/33; A61K 35/28; A61K 35/32; A61K 45/06

(52) U.S. Cl. CPC A61K 35/17; A61K 35/33; A61K 45/06; A61K 35/32; A61K 35/28

(57) ABSTRACT

Embodiments of the disclosure encompass T-regulatory cells for use in treating disc degenerative disease. The T-regulatory cells may be autologous or allogeneic to the individual being treated. In specific embodiments, the T-regulatory cells express certain markers and are derived from certain sources.

This application claims priority to U.S. Provisional Patent Application Ser. No. 62/666,807, filed May 4, 2018, which is incorporated by reference herein in its entirety.

### TECHNICAL FIELD

Embodiments of the disclosure concern at least the fields of cell biology, molecular biology, biochemistry, and medicine.

### BACKGROUND

In modern society, lower back pain is one of the most common disabilities and is the cause of significant physical and emotional discomfort. Deterioration of the structure of the intervertebral disc (IVD) is one of the leading causes of lower back pain. The disc is formed from a fibrous outer annulus fibrosus surrounding a softer, gelatinous nucleus pulposus. The fibers of the annulus fibrosus attach to the endplates of the vertebral bodies of the spinal cord and trap the nucleus pulposus, creating an isobaric environment. Under an axial load, the nucleus pulposus compresses and radially transfers that load to the annulus fibrosus. The laminated nature of the annulus fibrosus provides it with a high tensile strength and so allows it to expand radially in response to this transferred load. In a healthy IVD, the cells within the nucleus pulposus form only about one percent of the disc tissue by volume. These cells produce an extracellular matrix (ECM) containing a high percentage of proteoglycans. The proteoglycans contain sulfated functional groups that retain water, thereby providing the nucleus pulposus with its cushioning qualities. The nucleus pulposus cells may also secrete small amounts of cytokines and matrix metalloproteinases (MMPs), which help regulate the metabolism of the nucleus pulposus cells. In some instances of IVD disease, gradual degeneration of the IVD is caused by mechanical instabilities in other portions of the spine. In these instances, increased loads and pressures on the nucleus pulposus cause the cells within the disc (or invading macrophages) to emit larger than normal amounts of the above-mentioned cytokines. In other instances of IVD disease, genetic factors or apoptosis can cause a decline in the number of disc cells and/or a release of toxic amounts of cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to provide nutrients to the cells and eliminate waste may result in decreased cell viability and metabolism, resulting in further degradation of the ECM along with the accumulation of high levels of toxins that may cause nerve irritation and pain.

As IVD degeneration progresses, toxic levels of cytokines and MMPs present in the nucleus pulposus begin to degrade the ECM. In particular, MMPs (as mediated by cytokines) begin cleaving the water-retaining portions of the proteoglycans, thereby reducing its water-retaining capabilities. This degradation leads to a less flexible nucleus pulposus, which changes the loading pattern within the disc, and in turn may lead to delamination of the annulus fibrosus. These changes cause more mechanical instability, which can cause the cells to emit even more cytokines and lead to upregulation of MMPs. As this destructive cascade continues and IVD degeneration progresses, the disc begins to bulge (“a herniated disc”), and then ultimately ruptures, causing the nucleus pulposus to contact the spinal cord and produce pain.

Presently, the primary therapies for IVD degeneration are surgical interventions in which degenerated discs are excised or fused with neighboring discs. Surgical therapies aim to alleviate pain and other symptoms of IVD degeneration, but do nothing to repair or regenerate diseased IVDs. One approach for treating degenerated IVD cells and tissues is the use of cell-based therapies, in which living cells are administered to repair, replace, and/or remodel diseased tissues. Several recent studies have investigated the use of cell-based therapies for degenerative IVD conditions. For example, U.S. Pat. No. 6,352,557 (“Ferree”) and U.S. Pat. No. 6,340,369 (“Ferree II”) concern harvesting live IVD cells from a patient, culturing the cells and transplanting them into an affected IVD. Similarly, Alini, Eur. Spine J., 2002: 11(Supp. 2): S215-220, describes isolating and culturing cells from the nucleus pulposus, embedding the cells in a biomatrix, and then injecting the embedded cells into patients to restore functionality to affected IVDs. These approaches, while promising, have shown limited effectiveness in repairing degenerated IVDs and suffer from complications caused by immunological incompatibility between cell donors and recipients.

Unfortunately, numerous clinical trials using cellular therapy have not resulted in meaningful or reproducible results. One potential reason for this may be because of underlying inflammation in the IVD that decreases the ability of stem cells to function. The present disclosure provides a solution to this long-felt need in the art.

### BRIEF SUMMARY

The present disclosure is directed to methods and compositions for use in treating or preventing one or more disc diseases in a mammal, including intervertebral disc disease or degenerative disc disease, for example. The individual mammal may be a human, horse, dog, and so forth.

The present disclosure provides means of using T-regulatory cells (Treg or Tregs) administered intradiscally or in some embodiments intravenously or intra-arterially, for stimulation of IVD regeneration, as well as allowing for increased efficacy of other cell-based therapies, such as mesenchymal- or fibroblast-based therapies. In specific embodiments, the Tregs reduce inflammation, including in the disc.

In one embodiment, there is a method of treating or preventing disc degenerative disease in an individual, comprising the step of administering to the individual an effective amount of a plurality of T-regulatory cells. The T-regulatory cells may be autologous or allogeneic with respect to the recipient individual. In specific embodiments, the T-regulatory cells express a) CD4; b) CD25; c) CD73; d) CD105; e) LAP; f) TGF-beta; g) CTLA-4; h) GITR ligand; i) neuropilin-1; j) CTLA-4; k) FoxP3; l) CD127; m) GARP, or n) a combination thereof. The T-regulatory cells may be derived from cord blood, bone marrow, adipose stromal vascular fraction, peripheral blood (including peripheral blood that is mobilized peripheral blood), or a combination thereof. In specific cases, the T-regulatory cells are generated from T-regulatory cell progenitors, and the T-regulatory cell progenitors may be converted to T-regulatory cells by culture in a media comprising one or more reagents selected from the group consisting of a) anti-CD3; b) anti-CD28; c) TGF-beta; d) IL-10; f) rapamycin; g) TGF-beta; h) interleukin-2; i) VEGF, and (j) a combination thereof. [0010]In certain cases, the media comprises fibroblasts, mesenchymal stem cells, or both.

The T-regulatory cells in culture may be activated by one or more factors and/or one or more conditions, such as activated by exposure to vasoactive intestinal peptide, IL-10, TGF-beta, mesenchymal stem cell conditioned media, mesenchymal stem cell derived exosomes, BDNF, human chorionic gonadotropin, VEGF, hypoxic conditions, rapamycin, and/or angiopoietin that may be present alone in the media and/or may be produced by cells in the media

In at least some cases, the T-regulatory cells are anergic T cells.

The T-regulatory cells may be administered to the individual in any suitable route, including systemically or locally, such as intradiscally, for example. In specific embodiments, the T-regulatory cells are administered to the individual with an effective amount of fibroblasts, mesenchymal stem cells, nucleus pulposus cells, notochord cells, chondrocytes, or a combination thereof. Any of the cells may express one or more certain markers and/or may lack expression of one or more certain markers. The individual may be administered another therapy or preventative for disc degenerative disease, such as proper ergonomics and posture, pain relief, surgery, exercise, Chiropractic manipulation, massage, or a combination thereof, for example. The individual may be at risk for having a disc disease, such as an individual with a personal or family history, an athlete or former athlete, an individual whose vocation requires physical labor or required physical labor, and so forth.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 provides expression of FoxP3 in cells gated for CD4 and CD25 that were assessed by flow cytometry and expressed as percentage values. Blue Bars (on the left in the groupings of four bars) are MSC from Placenta, orange Bars (second from left) are MSC from Bone Marrow, grey Bars (second from right) are fibroblasts from skin, and dark yellow Bars (on the right) are fibroblasts from placenta.

FIG. 2 shows inhibition of TNF-alpha-induced apoptosis was achieved by addition of cells possessing the Treg CD4+ CD25+ phenotype. Blue bars (on the left in the bar groupings of four) are mononuclear cells, orange bars (second from left) are CD4 cells that are not fractionated, grey bars (second from right) are CD4 positive CD25 negative cells, and dark yellow bars (on the right) are CD4 positive, CD25 positive cells.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The term “administered” or “administering”, as used herein, refers to any method of providing a composition to an individual such that the composition has its intended effect on the individual. For example, one method of administering is by a direct mechanism such as local tissue administration (including by injection), oral ingestion, transdermal patch, topical, inhalation, suppository etc.

As used herein, “allogeneic” refers to tissues or cells from another body that in a natural setting are immunologically incompatible or capable of being immunologically incompatible, although from one or more individuals of the same species.

As used herein, the term “allotransplantation” refers to the transplantation of organs, tissues, and/or cells from a donor to a recipient, where the donor and recipient are different individuals, but of the same species. Tissue transplanted by such procedures is referred to as an allograft or allotransplant.

As used herein, the terms “allostimulatory” and “alloreactive” refer to stimulation and reaction of the immune system in response to an allologous antigens, or “alloantigens” or cells expressing a dissimilar HLA haplotype.

As used herein, “autologous” refers to tissues or cells that are derived or transferred from the same individual's body.

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

The term “biologically active” refers to any molecule having structural, regulatory or biochemical functions.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂, although in other cases these are altered. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “drug”, “agent” or “compound” as used herein, refers to any pharmacologically active substance capable of being administered that achieves a desired effect. Drugs or compounds can be synthetic or naturally occurring, non-peptide, proteins or peptides, oligonucleotides, or nucleotides (DNA and/or RNA), polysaccharides or sugars.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject. Methods of the disclosure include the reduction in intensity of disc degeneration and may include the reduction in intensity of one or more symptoms related thereto, including further degeneration, pain, and so forth.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. T-regulatory Cells and Preparation Thereof

The disclosure concerns the previously unknown finding that administration of T-regulatory cells (including intradiscally) stimulates proliferation of nucleus pulposus cells, at least. In specific cases, the disclosure encompasses the use of T-regulatory cells generated from fibroblast co-culture, or co-culture with other cells possessing or induced to possess immune suppressive activity. Other cells useful for generation of T regulatory cells include immature dendritic cells and/or immature monocytes. In one embodiment, allogeneic T-regulatory cells are administered together with allogeneic fibroblasts into an individual in need of treatment. In other embodiments, autologous T-regulatory cells are administered together with allogeneic fibroblasts, or vice versa, such as intradiscally.

In specific embodiments, the disclosure encompasses treatment of disc degenerative disease or prevention of disc degenerative disease in an individual at risk for disc degenerative disease (an individual at risk is an individual over the age of about 40, 45, 50, 55, 60, 65, 70, 75, 80, and so forth; an individual that is or was an athlete; an individual with a vocation that requires physical activity; an individual with a spinal injury; or a combination thereof, for example). A delay in the onset of disc degenerative disease, and/or the lessening of the severity of the disease, may be achieved utilizing methods encompassed herein.

The disclosure provides methods and compositions for treating disc degenerative disease using T-regulatory cells, such as through intradiscal administration of T-regulatory cells. T-regulatory cells may be of any kind. Specific T-regulatory cells for use in the current disclosure can be isolated from numerous known tissues, including peripheral blood [1], adipose stromal vascular fraction [2-5], cord blood [6-10], bone marrow [11], and/or mobilized peripheral blood [12], as examples.

The T-regulatory cells may be expanded using protocols known in the art. In some embodiments, methods for expanding T-regulatory cells are used. In some embodiments, the method comprises culturing T-regulatory cells with particular compositions, optionally for a certain duration of time. In particular cases, T-regulatory cells are cultured for at least 1, 2, 3, 4, 5, 6, 7, or more days. In addition, or alternatively, the T-regulatory cells are cultured under a certain level of oxygen and/or in the presence of certain cytokine(s) and/or in the presence of certain antibodies. In specific cases, the culture has the following conditions: 0.5-5% oxygen, 5-100 U/ml of IL-2, and/or the presence of anti-CD3 and anti-CD28 antibodies (used to expand and/or endow upregulation of immune suppressive activity of the Treg). In some embodiments TGF-beta may be added optionally.

In some embodiments, the T-regulatory cells to be expanded are CD4 positive and/or CD25 positive. In a plurality of T-regulatory cells, in some embodiments at least 90, 91, 92, 93, 94, 95, 96, 97, 98, or 99% of the T-regulatory cells are CD4 positive and CD25 positive T-regulatory cells and FoxP3 positive. In some embodiments, the T-regulatory cells comprise human cells. In some embodiments, the T-regulatory cells are cultured under 1% oxygen, such as no more than or at least 0.1, 0.2, 0.3, 0.4, 0.5, 0.6, 0.7, 0.8, or 0.9% oxygen.

In some embodiments, T-regulatory cells are isolated (e.g., from a culture medium) after culturing. In some embodiments, T-regulatory cells are isolated after at least 1, 2, 3, 4, 5, 6, 7, 8, 9, or 10 or more days of culture. Additionally or alternatively, in some embodiments T-regulatory cells expressing increased CTLA-4 and/or increased IL-10 levels as compared to control T-regulatory cells are isolated (e.g., from the culture medium, and/or from cells not expressing increased CTLA-4 and/or IL-10 levels) and utilized in methods of the disclosure. In some embodiments, the T-regulatory cells are contacted with one or more agents that increase intracellular cyclic AMP (cAMP) levels. In specific embodiments, an agent that increases cAMP levels in the cells may be one or more G protein-coupled receptor ligands. Examples of G protein-coupled receptor ligands include GPCR83 and/or GPCR173.

In particular embodiments, T-regulatory cells, within the context of the present disclosure, encompass cells expressing a Foxp3 protein. Foxp3 is expressed by CD4+CD25+ Tregs, and gain-of-function, overexpression and analysis of Foxp3-deficient Scurfy (sf) mice show that Foxp3 is at least useful to the development and maintenance of murine Tregs. All naturally occurring murine CD4+CD25+ Treg cells express Foxp3. TGF-beta1 can convert naive CD4+CD25− T cells to CD4+CD25+ Tregs via induction of Foxp3. Unlike CTLA-4, GITR and CD25, murine Foxp3 mRNA expression appears stable irrespective of T cell activation. Various surface proteins (CTLA-4, GITR, LAG-3, neuropilin-1) and cytokines (TGF-beta, IL-10) are expressed by Tregs, and like sf mice, mice lacking TGF-beta, CTLA-4 or CD25 die from autoimmunity. Human X-linked neonatal diabetes mellitus, enteropathy and endocrinopathy (IPEX) syndrome results in most cases from mutations in the forkhead/winged-helix domain of FOXP3 that disrupt critical DNA interactions; in sf mice, a frameshift mutation results in a protein lacking the forkhead domain. More than 20 mutations of FOXP3 are reported in IPEX syndrome, and the syndrome is lethal if untreated. By contrast, overexpression of murine Foxp3 gene leads to hypocellular peripheral lymphoid tissues with fewer T cells and a hypoactive immune state. Hence, control of Foxp3 levels within a certain range is useful for optimal immune functions and survival.

In one embodiment of the disclosure, T regulatory cells are obtained as follows. Cryopreserved cord blood bags (1 unit bags) are thawed and washed in CliniMACS buffer (Miltenyi Biotec, Bergish Gladbach, Germany) containing 0.5% HSA (Baxter Healthcare, Westlake Village, Calif.) in order to purify mononuclear cells. Subsequently, CD25⁺ cell enrichment is performed by positive selection using magnetic activated cell sorting (MACS) according to manufacturer's instructions (Miltenyi Biotec, Bergish Gladbach, Germany). Cells are checked for viability and subsequently stimulated by co-cultured with CD3/28 co-expressing Dynabeads® (ClinExVivo™ CD3/CD28, Invitrogen Dynal AS, Oslo, Norway) at a 1 cell: 3 bead ratio and re-suspended at 1×10⁶ cells/ml in X-VIVO 15 medium (Cambrex BioScience, Walkersville, Md.) supplemented with 10% human AB serum (Gemini Bio-Products, Sacramento, Calif.), 2 mM L-glutamine (Sigma, St. Louis, Mo.), 1% Penicillin-Streptomycin (Gibco/Invitrogen, Grand Island, N.Y.)] and 200 IU/ml interleukin (IL)-2 (CHIRON Corporation, Emeryville, Calif.). Ex vivo co-culture of the CD25⁺ cells and beads is performed in tissue culture flasks at 37° C. in a 5% CO₂-in-air atmosphere. The CB-derived CD25⁺ enriched T-cells are maintained at 1×10⁶ cells/ml by the addition of fresh medium and IL-2 (maintaining 200 IU/ml) every 48-72 hours.

T-regulatory cells may be isolated and enriched for CD4 positive, CD25 positive and optionally CD127 positive cells. By way of example, but not by way of limitation, in some embodiments, human peripheral blood mononuclear cells are separated from peripheral blood by density centrifugation using Ficoll. In some embodiments, peripheral blood mononuclear cells are labeled with anti-CD4, anti-CD25 and anti-CD127 antibodies and CD4 positive, CD25 med-hi, CD127 low cells are isolated as Treg by, e.g., FACS Aria II Cell Sorter. In some embodiments, cells are further enriched for FoxP3. In some embodiments, about 85%, 86%, 87%, 88%, 89%, 90%, 91%, 92%, 93%, 94%, 95%, 96%, 97%, 98%, 99% of the isolated and enriched Treg cells are CD4 positive and CD25 positive. In some embodiments, about 93% or greater, e.g., about 94%, 95%, 96%, 97%, 98%, 99% of the isolated and enriched Treg cells are CD4 positive and CD25 positive. In some embodiments, about 85%, 86%, 87%, 88%, 89%, 90%, 91%, 92%, 93%, 94%, 95%, 96%, 97%, 98%, 99% of the isolated and enriched CD4 positive, CD25 positive cells are FoxP3 positive. In some embodiments, about 95% of the isolated and enriched CD4 positive, CD25 positive cells are FoxP3 positive.

In some embodiments, a method for expanding T-regulatory cells is provided, including for use in intradiscal T-regulatory cell injection, as an example. In some embodiments, the method includes (a) culturing T-regulatory cells under normoxic conditions; and (b) culturing the T-regulatory cells of step (a) for at least 3 days under a certain percentage of oxygen, in the presence of one or more cytokines, and optionally in the presence of one or more antibodies. In specific embodiments the conditions in step (b) comprise the following conditions: 0.5-5% oxygen, 5-100 U/ml of IL-2, and in the presence of anti-CD2 and anti-CD28 antibodies.

In some embodiments, the T-regulatory cells to be expanded are CD4 positive and CD25 positive. In some embodiments, at least 90% of the CD4 positive and CD25 positive cells are FoxP3 positive. In some embodiments, the T-regulatory cells comprise human cells. In some embodiments, the T-regulatory cells are cultured under 1% oxygen. In some embodiments, the T-regulatory cells are contacted with an agent that increases intracellular cyclic AMP (cAMP) levels.

In some aspects, a method for expanding T-regulatory cells is provided. In some embodiments, the method includes culturing T-regulatory cells for at least 3 days in the presence of one or more agents that increase intracellular cyclic AMP (cAMP) levels. In some embodiments, the agent that increases intracellular cAMP levels includes one or more G protein-coupled receptor ligands. In some embodiments, the G protein-coupled receptor ligand includes one or more of: ligands of the A2A and A2B receptor (adenosine), ligands of the beta-adrenergic receptor ligands (adrenaline), ligands of D1 and D5 receptors (dopamine), ligands of H2 receptor (histamine), ligands of DP, IP, EP2 and EP4 receptors (prostaglandins), ligands of 5-HT4, 5-HT6, 5-HT7 receptors (serotonin), ligands of PACT, VPAC1, VPAC2 and/or ligands of glucagon receptors (VIP, PACAP, glucagon). In some embodiments, the compound that increases intracellular cAMP levels includes one or more of phosphodiesterase inhibitors, ibudilast, cholera toxin, forskolin, caffeine, theophylline, bucladesine, dibutyryl cAMP, db cAMP, pertussis toxin, milrinone, inamrinone, sildenafil, tadalafil, and/or activators of Gs protein. In some embodiments, the T-regulatory cells comprise human cells.

In particular embodiments the T-regulatory cells are capable of suppressing proliferation of activated T cells and/or are capable of suppressing production of IL-17. The T-regulatory cells may be derived from cord blood, bone marrow, peripheral blood and/or from mobilized peripheral blood. In cases when the T-regulatory cells are obtained from mobilized peripheral blood, it may be obtained from an individual that has been treated with G-CSF, that has been treated with Mozibil®, and/or that has been treated with GM-CSF.

In specific embodiments, T-regulatory cells are generated from T-regulatory cell progenitors, and in some cases, the T-regulatory cell progenitors are converted to T-regulatory cells by culture in a media comprising one or more reagents selected from the group consisting of a) anti-CD3; b) anti-CD28; c) TGF-beta; d) IL-10; f) rapamycin; g) TGF-beta; h) interleukin-2; i) VEGF, and (j) a combination thereof. In the culture, fibroblasts may be utilized with the T-regulatory cells. In specific embodiments, fibroblasts and/or mesenchymal stem cells are utilized in the culture to enhance generation of T-regulatory cells.

The T-regulatory cells may be activated by exposure to vasoactive intestinal peptide, IL-10, TGF-beta, mesenchymal stem cell conditioned media, mesenchymal stem cell derived exosomes, BDNF, human chorionic gonadotropin, VEGF, hypoxic conditions, rapamycin, and/or angiopoietin. In specific embodiments the activation occurs in culture prior to administration of the T-regulatory cells to the individual.

In some embodiments, the T-regulatory cells are activated by hypoxic conditions. In particular embodiments, the hypoxic condition comprises oxygen levels from 0.1%-10%, 0.1%-5%, 0.1%-2.5%, 0.1%-2%, 0.1%-1%, 0.5%-10%, 0.5%-7.5%, 0.5%-5%, 0.5%-2.5%, 0.5%-2%, 0.5%-1%, 1%-10%, 1%-7.5%, 1%-5%, 1%-2.5%, 1%-2%, 2%-10%, 2%-7.5%, 2%-5%, 2%-2.5%, 5%-10%, 5%-7.5%, 5%-6%, or 7.5%-10% oxygen, as examples only. The duration of exposure, including with (but not limited to) these representative levels of oxygen may be for a duration of 30 minutes (min)-3 days, 30 min-2 days, 30 min-1 day, 30 min-12 hours (hrs), 30 min-8 hrs, 30 min-6 hrs, 30 min-4 hrs, 30 min-2 hrs, 30 min-1 hour (hr), 1 hr-3 days, 1 hr-2 days, 1 hr-1 day, 1-12 hrs, 1-8 hrs, 1-6 hrs, 1-4 hrs, 1-2 hrs, 2 hrs-3 days, 2 hrs-2 days, 2 hrs-1 day, 2 hrs-12 hrs, 2-10 hrs, 2-8 hrs, 2-6 hrs, 2-4 hrs, 2-3 hrs, 6 hrs-3 days, 6 hrs-2 days, 6 hrs-1 day, 6-12 hrs, 6-8 hrs, 8 hrs-3 days, 8 hrs-2 days, 8 hrs-1 day, 8-16 hrs, 8-12 hrs, 8-10 hrs, 12 hrs-3 days, 12 hrs-2 days, 12 hrs-1 day, 12-18 hrs, 12-14 hrs, 1-3 days, or 1-2 days, as examples only.

For generation of T-regulatory cells for intradiscal injection, provided herein are methods of inducing differentiation of regulatory T cells by administering to cells undergoing inflammation a composition comprising one or more histone deacetylase (HDAC) inhibitory agents. In some embodiments, the step of administering comprises administering to dendritic cells and naive CD4⁺ T cells a composition comprising one or more histone deacetylase (HDAC) inhibitory agents. The HDAC inhibitor agent(s) may be present during the culture period, in at least some cases. In some embodiments, the step of administering comprises administering a composition comprising one or more histone deacetylase (HDAC) inhibitory agents that act to promote Treg differentiation, such as in a Foxp3-dependent manner. In some embodiments, the step of administering comprises administering a composition selected from the group consisting of bacterial metabolites, short chain fatty acids, histone deacetylase (HDAC) inhibitors, analogs thereof, and a combination thereof. In some embodiments, the step of administering comprises administering a composition comprising short chain fatty acids. In some embodiments, the step of administering comprises administering short chain fatty acids selected from the group consisting of butyrate, propionate, succinate, formate, valproate, phenylbutyrate, L-lactate, 2-ethylbutyrate, isovalerate, isobutyric acid, valeric acid, acetate, analogs thereof, and a combination thereof.

Cell expansion for cells originating from any of the above-mentioned tissues above may occur in clean room facilities purpose built for cell therapy manufacture and meeting GMP clean room classification. In a sterile class II biologic safety cabinet located in a class 10,000 clean production suite, cells were thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot was used for all experiments. Cells are subsequently placed in a T-225 flask containing 45 mL of cDMEM and cultured for 24 hours at 37° C. at 5% CO₂ in a fully humidified atmosphere. This allowed the MSC to adhere. Non-adherent cells were washed off using cDMEM by gentle rinsing of the flask. This resulted in approximately 6 million cells per initiating T-225 flask. The cells of the first flask were then split into 4 flasks. Cells were grown for 4 days after which approximately 6 million cells per flask were present (24 million cells total). This scheme was repeated but cells were not expanded beyond 10 passages, and were then banked in 6 million cell aliquots in sealed vials for delivery. All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. Donor cells are collected in sterile conditions, shipped to a contract manufacturing facility, assessed for lack of contamination and expanded. The expanded cells are stored in cryovials of approximately 6 million cells/vial, with approximately 100 vials per donor. At each step of the expansion quality control procedures were in place to ensure lack of contamination or abnormal cell growth.

In some embodiments, the cells are subjected to one or more media compositions that comprises, consists of, or consists essentially of Roswell Park Memorial Institute (RPMI-1640), Dublecco's Modified Essential Media (DMEM), Eagle's Modified Essential Media (EMEM), Optimem, Iscove's Media, or a combination thereof.

In one embodiment of the disclosure, the cells are cultured ex vivo using means known in the art for preserving viability and proliferative ability of the cells. In specific embodiments, there may be modification of known culture techniques to achieve one or more desired effects for the cells, such as decrease visibility of fibroblasts to a recipient immune system, and so forth. In one embodiment, the cells are cultured in conditions that lack one or more xenogeneic components, such as fetal calf serum. In specific embodiments, the disclosure encompasses the substitution of fetal calf serum with human platelet rich plasma, platelet lysate, umbilical cord blood serum, autologous serum, and/or defined cytokine mixes as an additional feature, for example to reduce the immunogenicity of the cells.

### III. Methods of Use of T-Regulatory Cells

The clinical use of T-regulatory cells has been previously described by numerous investigators and the means of expansion of T-regulatory cells can be applied to methods and compositions of the current disclosure. Examples of previous studies include: Safinia et al. reported the manufacture of clinical grade Tregs from prospective liver transplant recipients via a CliniMACS-based GMP isolation technique and expanded using anti-CD3/CD28 beads, IL-2 and rapamycin. They showed the enrichment of a pure, stable population of Tregs (>95% CD4(+)CD25(+)FOXP3(+)), reaching adequate numbers for their clinical application. The protocol proved successful in, influencing the expansion of superior functional Tregs, as compared to freshly isolated cells, whilst also preventing their conversion to Th17 cells under pro-inflammatory conditions [13]. Given that disc degenerative disease is associated with increase IL-17 production [14-25], in particular embodiments intradiscally administered T-regulatory cells are not Th17 cells. Another study described the ex vivo expanded CD4+CD25+CD127− T-regulatory cells in the treatment of graft versus host disease (GvHD). The cells were sorted from buffy coats taken from two family donors, expanded ex vivo and transferred to respective recipients who suffered from either acute or chronic GvHD. The therapy allowed for significant alleviation of the symptoms and reduction of pharmacologic immunosuppression in the case of chronic GvHD, while in the case of grade IV acute GvHD it only transiently improved the condition [26]. Brunstein et al. established a method of CD4(+)CD25(+)FoxP3(+) T-regulatory cell enrichment from cryopreserved UCB followed by a 18 (+) 1-day expansion culture including anti-CD3/anti-CD28 antibody-coated beads and recombinant human interleukin-2. In a “first-in-human” clinical trial, they evaluated the safety profile of UCB Treg in 23 patients. Patients received a dose of 0.1-30×10(5)UCB Treg/kg after double UCB transplantation. The targeted Treg dose was achieved in 74% of cultures, with all products being suppressive in vitro (median 86% suppression at a 1:4 ratio). No infusional toxicities were observed. After infusion, UCB Treg could be detected for 14 days, with the greatest proportion of circulating CD4(+)CD127(−)FoxP3(+) cells observed on day (+)2. Compared with identically treated 108 historical controls without Treg, there was a reduced incidence of grade II-IV aGVHD (43% vs 61%, P=0.05) with no deleterious effect on risks of infection, relapse, or early mortality [27]. In another study in the area of type 1 diabetes administered Tregs in 10 type 1 diabetic children (aged 8-16 years) within 2 months since diagnosis. In total, 4 patients received 10×10(6) Tregs/kg body wt, and the remaining 6 patients received 20×10(6) Tregs/kg body wt. The preparation consisted of sorted autologous CD3(+)CD4(+)CD25(high)CD127(−) Tregs expanded under good manufacturing practice conditions. No toxicity of the therapy was noted. A significant increase in the percentage of Tregs in the peripheral blood has been observed since the day of infusion. These patients were followed along with matched type 1 diabetic patients not treated with Tregs. Half a year after type 1 diabetes onset (4-5 months after Tregs infusion), 8 patients treated with Tregs still required <0.5 UI/kg body wt of insulin daily, with 2 patients out of insulin completely, whereas the remission was over in the nontreated group. In addition, plasma C-peptide levels were significantly higher in the treated group as compared with those not treated [28].

In some embodiments, the T-regulatory cells with or without other cells are administered (e.g., injected) into the disc through a needle, such as a small bore needle. Alternatively, the formulation can also be injected into the disc using the same small bore needle. In some embodiments, the needle has a bore of about 22 gauge or less, so that the possibilities of producing a herniation are mitigated. For example, the needle can have a bore of about 24 gauge or less, so that the possibilities of producing a herniation are even further mitigated. If the volume of the direct injection of the cells or formulation is sufficiently high so as to cause a concern of over-pressurizing the nucleus pulposus, then at least a portion of the nucleus pulposus may be removed prior to administration (i.e., direct injection) of dedifferentiated autologous or allogeneic fibroblasts. In some embodiments, the volume of removed nucleus pulposus is substantially similar to the volume of the formulation to be injected. For example, the volume of removed nucleus pulposus can be within about 80-120% of the volume of the formulation to be injected. In addition, this procedure has the added benefit of at least partially removing some degenerated disc from the patient. When injecting the T-regulatory cells into the nucleus pulposus, it is useful that the volume of drug (i.e., formulation of cells suspended in growth medium or a carrier) delivered be between about 0.5 ml and about 3.0 ml comprising cells suspended in growth medium or a carrier. When injected in these smaller quantities, it is believed that the added or replaced volume will not cause an appreciable pressure increase in the nucleus pulposus. Factors to consider when determining the volume of drug to be delivered include the size of the disc, the amount of disc removed and the concentration of the T-regulatory cells in the growth medium or carrier.

In some embodiments, the T regulatory cells are exposed in culture to de-differentiated fibroblasts and/or dedifferentiated fibroblasts are delivered to the individual in addition to the T regulatory cells being delivered to the individual. The dedifferentiated fibroblast cells may have been produced upon exposure to stem cells and/or cytoplasm from stem cells, including pluripotent stem cells selected from the group consisting of a) parthenogenic stem cells; b) embryonic stem cells; c) inducible pluripotent stem cells; d) somatic cell nuclear transfer derived stem cells; e) Stimulus-triggered acquisition of pluripotency (STAP); and f) a combination thereof. The de-differentiated fibroblasts may have been produced upon exposure to hypoxia; one or more histone deacetylase inhibitors (such as selected from the group consisting of a) valproic acid; b) trichostatin A; c) phenylbutyrate; d) vorinostat; e) belinostat; f) LAQ824; g) panobinostat; h) entinostat; i) CI994; j) mocetinostat; k) sulforaphane; and l) a combination thereof); one or more DNA methyltransferase inhibitors (such as selected from the group consisting of a) decitabine; b) 5-azacytidine; c) Zebularine; d) RG-108; e) procaine hydrochloride; f) Procainamide hydrochloride; g) Hydralazine hydrochloride; h) Epigallocatechin gallate; i) Chlorogenic acid; j) Caffeic acid; and h) a combination thereof); and/or 2%-8%, 2%-7%, 2%-6%, 2%-5%, 2%-4%, 2%-3%, 3%-8%, 3%-7%, 3%-6%, 3%-5%, 3%-4%, 4%-8%, 4%-7%, 4%-6%, 4%-5%, 5%-8%, 5%-7%, 5%-6%, 6%-8%, 6%-7%, or 7%-8% oxygen.

In one embodiment, there is a method for treating disc degenerative disease comprising the steps of a) optionally obtaining a population of T-regulatory cells; and b) administering to an individual T-regulatory cells at a concentration and frequency (such as intradiscally) sufficient to induce regeneration of the degenerated disc. The T-regulatory cell may be autologous or allogeneic with respect to the individual in need of treatment. In specific embodiments, the T-regulatory cells express one or more markers selected from the group consisting of a) CD4; b) CD25; c) CD73; d) CD105; e) LAP; f) TGF-beta; g) CTLA-4; h) GITR ligand; i) neuropilin-1; j) CTLA-4; k) FoxP3; l) CD127; m) GARP, and n) a combination thereof.

For treatment of disc disease(s), a suitable amount of T-regulatory cells are provided to an individual. The T-regulatory cells may be administered intradiscally, in particular cases, and in some embodiments they are administered at the same time(s) or different time(s) as fibroblasts, mesenchymal stem cells, nucleus pulposus cells, notochord cells, chondrocytes, adipose stromal vascular fraction cells, or a combination thereof. In specific cases, T-regulatory cells possess the ability to inhibit proliferation of naïve T cells stimulated with a signal that activates proliferation, and the signal that activates proliferation may be selected from the group consisting of a) anti-CD3 and anti-CD28 beads; b) concanavalin A; c) PHA; d) stimulation with alloreactive antigen presentation cells; and e) a combination thereof. In specific cases, the T-regulatory cell is capable of suppressing maturation of dendritic cells, and in specific cases the dendritic cell maturation comprises upregulation of CD40, CD80, and/or CD86 in dendritic cells.

In cases wherein T-regulatory cells and other types of cells are administered to an individual, the administrations may be concurrent or in succession. In cases wherein the T-regulatory cells are administered to the individual at a different time than other types of cells, the T-regulatory cells may be administered before and/or after the other types of cells. In cases wherein there are multiple administrations of T-regulatory cells to an individual, the different administrations may comprise different types of other cells. For example, in one case Tregs are provided in addition to fibroblasts but in another administration Tregs are provided in addition to chondrogenic cells.

In cases wherein fibroblasts are utilized in methods of the disclosure, either to prepare the T-regulatory cells or to use for administration to an individual, the fibroblasts may be selected from the group consisting of adult fibroblasts, embryonic fibroblasts, fetal fibroblasts, placental fibroblasts, adipose stromal derived fibroblasts, adipose-derived Stromal Vascular Fraction (SVF) fibroblasts, and combinations thereof. In certain embodiments, fibroblasts may be derived from tissues comprising skin, heart, blood vessels, bone marrow, skeletal muscle, liver, pancreas, brain, adipose tissue, foreskin, placental, and/or umbilical cord, for example. In specific embodiments, the fibroblasts are placental, fetal, neonatal or adult or mixtures thereof.

In some cases, T cells are engineered to express genes associated with regeneration of nucleus pulposus cells including BMP, FGF, EGF, and/or PDGF.

The number of administrations of T-regulatory cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

### IV. Kits of the Disclosure

Any of the cellular and/or non-cellular compositions described herein or similar thereto may be comprised in a kit. In a non-limiting example, one or more reagents for use in methods for preparing T-regulatory cells may be comprised in a kit. Such reagents may include cells, vectors, one or more growth factors, vector(s) one or more costimulatory factors, media, enzymes, buffers, nucleotides, salts, primers, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate particular embodiments of the disclosure. It should be appreciated by those of skill in the art that the techniques disclosed in the examples that follow represent techniques discovered by the inventor to function well in the practice of the methods of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Intradiscal T Regulatory Cell Administration For Treatment of Disc Degenerative Disease

### Generation of T Regulatory Cells

Human dermal fibroblasts or placental fibroblasts were plated at 1.3×10⁵ per centimeter square in Dulbecco's modified Eagle's medium-low glucose supplemented with 10% fetal calf serum and Penicillin/Streptomycin. After 3-4 days, nonadherent cells were removed and medium was refreshed every 3-4 days until cells reached approximately 90% confluence. The fibroblast monolayer was detached using trypsin/EDTA (Invitrogen Corp.) and reseeded at 4,000 per cm² for further expansion. The fibroblasts where used in the experiments at passage 2-5. For controls, mesenchymal stem cells from bone marrow and placenta were also used. Peripheral blood mononuclear cells were obtained from AllCells (Leuko Pak), thawed and washed in phosphate buffered saline (PBS) twice by centrifugation at 400 g for 10 minutes.

CD4⁺ T cells were isolated from PBMC by magnetic activated cell sorting (MACS) using a cocktail of biotin-labeled antibodies against CD8, CD14, CD16, CD19, CD36, CD56, CD123, TCRγ/δ, and CD235a and anti-biotin microbeads followed by separation on a MACS LS column according to the manufacturer's recommendations. 200,000 CD4 cells where cultured at various ratios with fibroblasts or MSC in RPMI media supplemented with fetal calf serum for 7 days. As observed, an increase in T cells possessing T regulatory cell phenotype (CD4+ CD25+ FoxP3+) was seen in CD4 cells cultured with fibroblasts. Interestingly, coculture with mesenchymal stem cells (bone marrow MSC, purchased from AllCells, placental MSC (gift from Weiping Min Laboratory, University of Western Ontario, Canada), resulted in less generation of CD4+ CD25+ FoxP3+ cells.

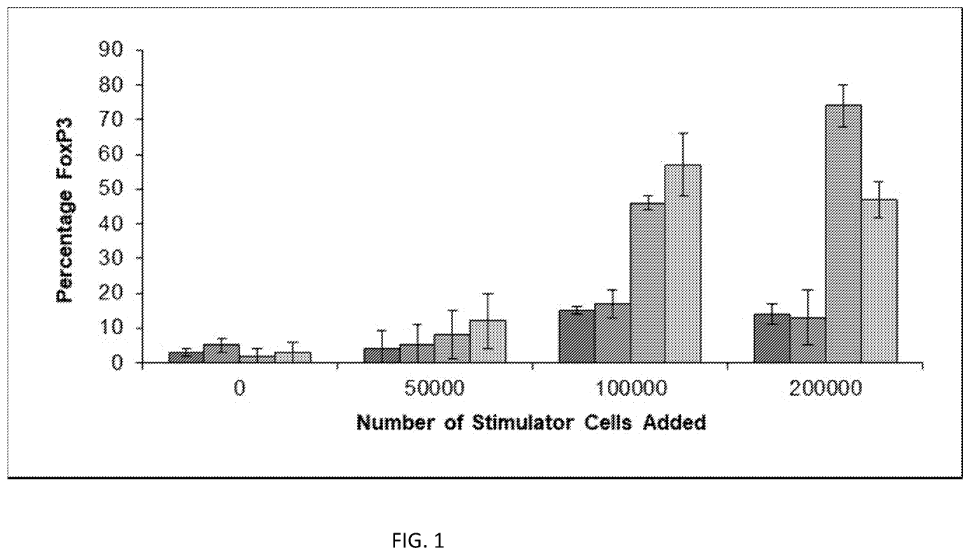

Expression of FoxP3 in cells gated for CD4 and CD25 was assessed by flow cytometry and expressed as percentage values in FIG. 1. Blue Bars are MSC from Placenta, Orange Bars are MSC from Bone Marrow, Grey Bars are fibroblasts from skin, and Yellow Bars are fibroblasts from placenta. A higher percentage of FoxP3 expressing cells were found in cultures with Fibroblasts as compared to MSC.

### Example 2

### Inhibition of Apoptosis of Nucleus Pulposus Cells by Treg Cells

Nucleus pulposus (NP) tissue samples were obtained from patients undergoing spine surgery because of burst thoracolumbar fracture. The experimental protocol was approved by the institutional review board with informed consent from the patients. The NP tissues were treated with 0.25% pronase (Sigma-Aldrich, Louis, Mo., USA) for 30 min. and 0.2% collagenase type II (Invitrogen, Carlsbad, Calif., USA) for 4 hrs at 37° C. The digest was filtered through a 70-μm pore size mesh and then cultured in Dulbecco's modified Eagle's medium (DMEM; Gibco, Grand Island, N.Y., USA) with 10% fetal bovine serum (FBS; Invitrogen), 1% penicillin-streptomycin (Sigma-Aldrich), 2 mM glutamine (Sigma-Aldrich) and 50 μg/mL L-ascorbic acid (Sigma-Aldrich) in T25 flasks at 37° C. in 5% CO₂. When grew to confluence, the cells were digested by 0.25% trypsin/1 mM EDTA and passed into bigger flasks for expansion. The nucleus pulposus cells (NPCs) from passage 4 or 5 were plated into experimental plates for all of the experiments.

NPCs were plated into 6-well plates at a density of 1×10⁵ cells per well. To test the apoptosis-inducing effect of TNF-α on cells, NPCs were treated with 0, 5, 10 or 30 ng/ml TNF-α (Sigma-Aldrich) for 12 hrs. To assess the anti-apoptotic effect of added cells, control non-expanded mononuclear cells, CD4 positive purified cells, CD4 positive CD25 negative and CD4 positive CD25 positive cells where added to the cultures of NPC subsequent to TNF treatment. Cells where cultured for an additional 24 hours. Concentration of lymphocytes added was 1×10⁵ cells per well.

NPC apoptosis rates were evaluated by flow cytometry using an Annexin V/PI apoptosis detection kit (BD Biosciences, Franklin Lakes, N.J., USA). NPCs were washed twice with PBS, resuspended in binding buffer and incubated with 5 μl FITC-Annexin V and 5 μl PI for 15 min. at room temperature. Staining cells were analyzed using the FACScan flow cytometry system (Becton Dickinson, San Diego, Calif., USA).

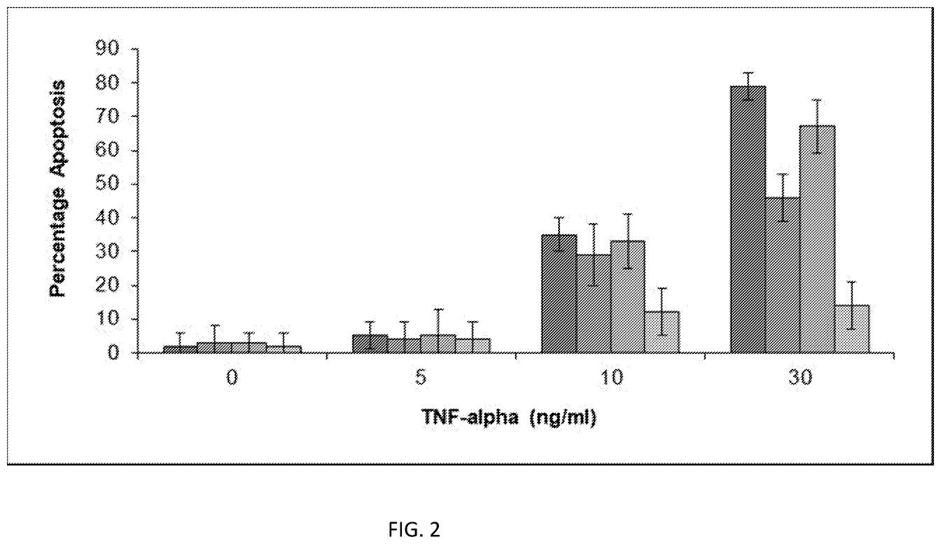

As seen in FIG. 2, the greatest inhibition of TNF-alpha-induced apoptosis was achieved by addition of cells possessing the Treg CD4+ CD25+ phenotype. Blue bars are mononuclear cells, orange bars are CD4 cells that are not fractionated, grey bars are CD4 positive CD25 negative cells, and dark yellow bars are CD4 positive, CD25 positive cells.

### REFERENCES

All patents publications mentioned in the specification are indicative of the level of those skilled in the art to which the invention pertains. All patents and publications are herein incorporated by reference to the same extent as if each individual publication was specifically and individually indicated to be incorporated by reference.

1. Ramlal, R. and G. C. Hildebrandt, Advances in the Use of Regulatory T-Cells for the Prevention and Therapy of Graft-vs.-Host Disease. Biomedicines, 2017. 5(2).

2. Ichim, T. E., et al., Autologous stromal vascular fraction cells: a tool for facilitating tolerance in rheumatic disease. Cell Immunol, 2010. 264(1): p. 7-17.

3. Kalin, S., et al., A Stat6/Pten Axis Links Regulatory T Cells with Adipose Tissue Function. Cell Metab, 2017. 26(3): p. 475-492 e7.

4. Becker, M., M. K. Levings, and C. Daniel, Adipose-tissue regulatory T cells: Critical players in adipose-immune crosstalk. Eur J Immunol, 2017.

5. Pereira, S., et al., Modulation of adipose tissue inflammation by FOXP3+ Treg cells, IL-10, and TGF-beta in metabolically healthy class III obese individuals. Nutrition, 2014. 30(7-8): p. 784-90.

6. El-Chennawi, F., et al., Comparison of the percentages of CD4+ CD25high FOXP3+, CD4+ CD25low FOXP3+, and CD4+ FOXP3+ Tregs, in the umbilical cord blood of babies born to mothers with and without preeclampsia. Am J Reprod Immunol, 2017.

7. Seay, H. R., et al., Expansion of Human Tregs from Cryopreserved Umbilical Cord Blood for GMP-Compliant Autologous Adoptive Cell Transfer Therapy. Mol Ther Methods Chin Dev, 2017. 4: p. 178-191.

8. Parmar, S. and E. J. Shpall, Treg adoptive therapy: is more better? Blood, 2016. 127(8): p. 962-3.

9. Brunstein, C. G., et al., Umbilical cord blood-derived T-regulatory cells to prevent GVHD: kinetics, toxicity profile, and clinical effect. Blood, 2016. 127(8): p. 1044-51.

10. Parmar, S., et al., Ex vivo fucosylation of third-party human regulatory T cells enhances anti-graft-versus-host disease potency in vivo. Blood, 2015. 125(9): p. 1502-6.

11. Zhao, X. Y., et al., Higher frequency of regulatory T cells in granulocyte colony-stimulating factor (G-CSF)-primed bone marrow grafts compared with G-CSF-primed peripheral blood grafts. J Transl Med, 2015. 13: p. 145.

12. Patel, P., et al., Clinical grade isolation of regulatory T cells from G-CSF mobilized peripheral blood improves with initial depletion of monocytes. Am J Blood Res, 2015. 5(2): p. 79-85.

13. Safinia, N., et al., Successful expansion of functional and stable regulatory T cells for immunotherapy in liver transplantation. Oncotarget, 2016. 7(7): p. 7563-77.

14. Scuderi, G. J., et al., Cytokine assay of the epidural space lavage in patients with lumbar intervertebral disk herniation and radiculopathy. J Spinal Disord Tech, 2006. 19(4): p. 266-9.

15. Shamji, M. F., et al., Proinflammatory cytokine expression profile in degenerated and herniated human intervertebral disc tissues. Arthritis Rheum, 2010. 62(7): p. 1974-82.

16. Gabr, M. A., et al., Interleukin-17 synergizes with IFNgamma or TNFalpha to promote inflammatory mediator release and intercellular adhesion molecule-1 (ICAM-1) expression in human intervertebral disc cells. J Orthop Res, 2011. 29(1): p. 1-7.

17. Gruber, H. E., et al., Increased IL-17 expression in degenerated human discs and increased production in cultured annulus cells exposed to IL-1ss and TNF-alpha. Biotech Histochem, 2013. 88(6): p. 302-10.

18. Risbud, M. V. and I. M. Shapiro, Role of cytokines in intervertebral disc degeneration: pain and disc content. Nat Rev Rheumatol, 2014. 10(1): p. 44-56.

19. Zhang, W., et al., Th17 cell frequency and IL-17 concentration correlate with pre- and postoperative pain sensation in patients with intervertebral disk degeneration. Orthopedics, 2014. 37(7): p. e685-91.

20. Wang, S. S., et al., IL-17A enhances ADAMTS-7 expression through regulation of TNF-alpha in human nucleus pulposus cells. J Mol Histol, 2015. 46(6): p. 475-83.

21. Zhang, Y., et al., Production of CCL20 on nucleus pulposus cells recruits IL-17-producing cells to degenerated IVD tissues in rat models. J Mol Histol, 2016. 47(1): p. 81-9.

22. Li, J. K., et al., IL-17 mediates inflammatory reactions via p38/c-Fos and JNK/c-Jun activation in an AP-1-dependent manner in human nucleus pulposus cells. J Transl Med, 2016. 14: p. 77.

23. Liu, X. G., H. W. Hou, and Y. L. Liu, Expression levels of IL-17 and TNF-alpha in degenerated lumbar intervertebral discs and their correlation. Exp Ther Med, 2016. 11(6): p. 2333-2340.

24. Hu, B., et al., Interleukin-17 upregulates vascular endothelial growth factor by activating the JAK/STAT pathway in nucleus pulposus cells. Joint Bone Spine, 2017. 84(3): p. 327-334.

25. Yao, Z., et al., Salubrinal Suppresses IL-17-Induced Upregulation of MMP-13 and Extracellular Matrix Degradation Through the NF-kB Pathway in Human Nucleus Pulposus Cells. Inflammation, 2016. 39(6): p. 1997-2007.

26. Trzonkowski, P., et al., First-in-man clinical results of the treatment of patients with graft versus host disease with human ex vivo expanded CD4+CD25+CD127− T-regulatory cells. Clin Immunol, 2009. 133(1): p. 22-6.

27. Brunstein, C. G., et al., Infusion of ex vivo expanded T-regulatory cells in adults transplanted with umbilical cord blood: safety profile and detection kinetics. Blood, 2011. 117(3): p. 1061-70.

28. Marek-Trzonkowska, N., et al., Administration of CD4+CD25highCD127− regulatory T cells preserves beta-cell function in type 1 diabetes in children. Diabetes Care, 2012. 35(9): p. 1817-20.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of treating or preventing disc degenerative disease in an individual, comprising the step of administered to the individual an effective amount of a plurality of T-regulatory cells.

2. The method of claim 1, wherein the T-regulatory cells are autologous or allogeneic with respect to the individual.

3. The method of claim 1, wherein the T-regulatory cells express a) CD4; b) CD25; c) CD73; d) CD105; e) LAP; f) TGF-beta; g) CTLA-4; h) GITR ligand; i) neuropilin-1; j) CTLA-4; k) FoxP3; l) CD127; m) GARP, or n) a combination thereof.

4. The method of claim 1, wherein the T-regulatory cells are derived from cord blood, bone marrow, adipose stromal vascular fraction, or peripheral blood.

5. The method of claim 4, wherein the peripheral blood is mobilized peripheral blood.

6. The method of claim 1, wherein the T-regulatory cells are generated from T-regulatory cell progenitors.

7. The method of claim 6, wherein the T-regulatory cell progenitors are converted to T-regulatory cells by culture in a media comprising one or more reagents selected from the group consisting of a) anti-CD3; b) anti-CD28; c) TGF-beta; d) IL-10; f) rapamycin; g) TGF-beta; h) interleukin-2; i) VEGF, and a combination thereof.

8. The method of claim 7, wherein the media comprises fibroblasts, mesenchymal stem cells, or both.

9. The method of claim 1, wherein the T-regulatory cells in culture are activated by one or more factors and/or one or more conditions.

10. The method of claim 9, wherein the T-regulatory cells are activated by exposure to vasoactive intestinal peptide, IL-10, TGF-beta, mesenchymal stem cell conditioned media, mesenchymal stem cell derived exosomes, BDNF, human chorionic gonadotropin, VEGF, hypoxic conditions, rapamycin, and/or angiopoietin.

11. The method of claim 1, wherein the T-regulatory cells are anergic T cells.

12. The method of claim 1, wherein the T-regulatory cells are administered to the individual intradiscally.

13. The method of claim 1, wherein the T-regulatory cells are administered to the individual with an effective amount of fibroblasts, mesenchymal stem cells, nucleus pulposus cells, notochord cells, chondrocytes, or a combination thereof.

14. The method of claim 13, wherein the fibroblasts are dedifferentiated fibroblasts.

15. The method of claim 1, wherein the individual is administered another therapy or preventative for disc degenerative disease.

16. The method of claim 15, wherein the other therapy is proper ergonomics and posture, pain relief, surgery, exercise, Chiropractic manipulation, massage, or a combination thereof.
